SEQUENCE LISTING (40 sequences)

SEQ ID NO: 1 - protein, 241 aa, Homo sapiens
AQSPKGSPLAPSGTSVPFWVRMSPEFVAVQPGKSVQLNCSNSCPQPQNSSLRTPLRQGKTLRGPGWVSYQLLDVRAWSSLAHCLVTCAGKTRWATSRITAYKPPHSVILEPPVLKGRKYTLRCHVTQVFPVGYLVVTLRHGSRVIYSESLERFTGLDLANVTLTYEFAAGPRDFWQPVICHARLNLDGLVVRNSSAPITLMLAWSPAPTALASGSIAALVGILLTVGAAYLCKCLAMKSQA

SEQ ID NO: 2 - protein, 11 aa, Homo sapiens
PFWVRMSPEFV

SEQ ID NO: 3 - protein, 11 aa, Homo sapiens
KTRWATSRITA

SEQ ID NO: 4 - protein, 11 aa, Homo sapiens
WSSLAHCLVTC

SEQ ID NO: 5 - protein, 11 aa, Homo sapiens
NVTLTYEFAAG

SEQ ID NO: 6 - protein, 11 aa, Homo sapiens
GRKYTLRCHVT

SEQ ID NO: 7 - protein, 8 aa, Homo sapiens
QNSSLRTP

SEQ ID NO: 8 - protein, 9 aa, Homo sapiens
LERFTGLDL

SEQ ID NO: 9 - protein, 9 aa, Artificial Sequence
  Description of Artificial Sequence Synthetic peptide
SVPFWVRMS

SEQ ID NO: 10 - protein, 9 aa, Artificial Sequence
  Description of Artificial Sequence Synthetic peptide
TRWATSRIT

SEQ ID NO: 11 - protein, 9 aa, Artificial Sequence
  Description of Artificial Sequence Synthetic peptide
AWSSLAHCL

SEQ ID NO: 12 - DNA, 723 bp, Homo sapiens
gcgcaaagccccaagggtagccctctcgcgccctccgggacctcagtgcccttctgggtgcgcatgagcccggagttcgtggctgtgcagccggggaagtcagtgcagctcaattgcagcaacagctgtccccagccgcagaattccagcctccgcaccccgctgcggcaaggcaagacgctcagagggccgggttgggtgtcttaccagctgctcgacgtgagggcctggagctccctcgcgcactgcctcgtgacctgcgcaggaaaaacacgctgggccacctccaggatcaccgcctacaaaccgccccacagcgtgattttggagcctccggtcttaaagggcaggaaatacactttgcgctgccacgtgacgcaggtgttcccggtgggctacttggtggtgaccctgaggcatggaagccgggtcatctattccgaaagcctggagcgcttcaccggcctggatctggccaacgtgaccttgacctacgagtttgctgctggaccccgcgacttctggcagcccgtgatctgccacgcgcgcctcaatctcgacggcctggtggtccgcaacagctcggcacccattacactgatgctcgcttggagccccgcgcccacagctttggcctccggttccatcgctgcccttgtagggatcctcctcactgtgggcgctgcgtacctatgcaagtgcctagctatgaagtcccaggcg

SEQ ID NO: 13 - protein, 9 aa, Artificial Sequence
  Description of Artificial Sequence Synthetic peptide
RQGKTLRGP

SEQ ID NO: 14 - DNA, 21 bp, Artificial Sequence
  Description of Artificial Sequence Synthetic oligonucleotide
agaacccactgcttactggct

SEQ ID NO: 15 - DNA, 20 bp, Artificial Sequence
  Description of Artificial Sequence Synthetic oligonucleotide
tgagcctgcttccagcagca

SEQ ID NO: 16 - DNA, 21 bp, Artificial Sequence
  Description of Artificial Sequence Synthetic oligonucleotide
tcagtgcccgcctgggtgcgc

SEQ ID NO: 17 - DNA, 21 bp, Artificial Sequence
  Description of Artificial Sequence Synthetic oligonucleotide
gcgcacccaggcgggcactga

SEQ ID NO: 18 - DNA, 21 bp, Artificial Sequence
  Description of Artificial Sequence Synthetic oligonucleotide
gtgcccttcgcggtgcgcatg

SEQ ID NO: 19 - DNA, 21 bp, Artificial Sequence
  Description of Artificial Sequence Synthetic oligonucleotide
catgcgcaccgcgaagggcac

SEQ ID NO: 20 - DNA, 21 bp, Artificial Sequence
  Description of Artificial Sequence Synthetic oligonucleotide
cccttctggacgcgcatgagc

SEQ ID NO: 21 - DNA, 21 bp, Artificial Sequence
  Description of Artificial Sequence Synthetic oligonucleotide
gctcatgcgcgtccagaaggg

SEQ ID NO: 22 - DNA, 20 bp, Artificial Sequence
  Description of Artificial Sequence Synthetic oligonucleotide
ggaaaaacagaatgggccac

SEQ ID NO: 23 - DNA, 20 bp, Artificial Sequence
  Description of Artificial Sequence Synthetic oligonucleotide
gtggcccattctgtttttcc

SEQ ID NO: 24 - DNA, 21 bp, Artificial Sequence
  Description of Artificial Sequence Synthetic oligonucleotide
acacgctggctcacctccagg

SEQ ID NO: 25 - DNA, 21 bp, Artificial Sequence
  Description of Artificial Sequence Synthetic oligonucleotide
cctggaggtgagccagcgtgt

SEQ ID NO: 26 - DNA, 20 bp, Artificial Sequence
  Description of Artificial Sequence Synthetic oligonucleotide
cgctgggccgtctccaggat

SEQ ID NO: 27 - DNA, 20 bp, Artificial Sequence
  Description of Artificial Sequence Synthetic oligonucleotide
atcctggagacggcccagcg

SEQ ID NO: 28 - DNA, 21 bp, Artificial Sequence
  Description of Artificial Sequence Synthetic oligonucleotide
tgggccaccgccaggatcacc

SEQ ID NO: 29 - DNA, 21 bp, Artificial Sequence
  Description of Artificial Sequence Synthetic oligonucleotide
ggtgatcctggcggtggccca

SEQ ID NO: 30 - DNA, 20 bp, Artificial Sequence
  Description of Artificial Sequence Synthetic oligonucleotide
gccacctccgagatcaccgc

SEQ ID NO: 31 - DNA, 20 bp, Artificial Sequence
  Description of Artificial Sequence Synthetic oligonucleotide
gcggtgatctgggaggtggc

SEQ ID NO: 32 - DNA, 20 bp, Artificial Sequence
  Description of Artificial Sequence Synthetic oligonucleotide
gggccgggtgcggtgtctta

SEQ ID NO: 33 - DNA, 20 bp, Artificial Sequence
  Description of Artificial Sequence Synthetic oligonucleotide
taagacaccgcacccggccc

SEQ ID NO: 34 - DNA, 20 bp, Artificial Sequence
  Description of Artificial Sequence Synthetic oligonucleotide
aagggcagggaatacacttt

SEQ ID NO: 35 - DNA, 20 bp, Artificial Sequence
  Description of Artificial Sequence Synthetic oligonucleotide
aaagtgtattccctgccctt

SEQ ID NO: 36 - DNA, 21 bp, Artificial Sequence
  Description of Artificial Sequence Synthetic oligonucleotide
gatctggccgccgtgaccttg

SEQ ID NO: 37 - DNA, 21 bp, Artificial Sequence
  Description of Artificial Sequence Synthetic oligonucleotide
caaggtcacggcggccagatc

SEQ ID NO: 38 - DNA, 20 bp, Artificial Sequence
  Description of Artificial Sequence Synthetic oligonucleotide
gccaacgtggtcttgaccta

SEQ ID NO: 39 - DNA, 20 bp, Artificial Sequence
  Description of Artificial Sequence Synthetic oligonucleotide
taggtcaagaccacgttggc

SEQ ID NO: 40 - DNA, 723 bp, Homo sapiens; identical to SEQ ID NO: 12
  CDS (1)..(723)

(19) United States
(12) Patent Application Publication
(10) Pub. No.: US 2006/0252116 A1
(43) Pub. Date: Nov. 9, 2006

(54) Icam-4 binding sites

(76) Inventors: Tosti Jon Mankelow, Bristol (GB); Frances Adrienne Spring, Bristol (GB); Stephen Frederick Parsons, Bristol (GB); David John Anstee, Bristol (GB)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/533,817

(22) PCT Filed: Nov. 4, 2003
(86) PCT No.: PCT/GB03/04744

§ 371 Date: Aug. 30, 2005

Related U.S. Application Data
(60) Provisional application No. 60/423,391, filed on Nov. 4, 2002.
(60) Provisional application No. 60/431,721, filed on Dec. 9, 2002.

(51) Int. Cl. C12P 21/06; C07H 21/04; C07K 14/74; A61K 38/17

(52) U.S. Cl. 435/69.1; 435/320.1; 435/325; 530/350; 514/2; 536/23.5

(57) ABSTRACT

The present invention relates to intercellular adhesion molecule-4 (ICAM-4), including binding sites on ICAM-4, antagonists affecting ICAM-4 and uses thereof. In one aspect of the invention there is provided an epitope for binding integrins, comprising strands A (or F) and G of domain 1 of ICAM-4. In another aspect of the invention there is provided a footprint domain for binding integrins, comprising a first epitope as defined above and second epitope comprising the C and F strands of domain 1 and the CE loop of domain 2 of ICAM-4.

The present invention relates to intercellular adhesion molecule-4 (ICAM-4). In particular, the invention relates to binding sites on ICAM-4, antagonists affecting ICAM-4 and uses thereof.

Intercellular adhesion molecule-4 (ICAM-4) is expressed chiefly on erythroid cells and is the glycoprotein that carries the LW blood group antigens. A study by Bailly et al. (1995, Eur. J. Inumunol. 25: 3316-3320) showed binding of integrins LFA-1 and Mac-1 (also known as αMβ2) to ICAM-4.

Another report shows that ICAM-4 binds hemopoietic (HEL) and non-hemopoietic (FLYRD18, a derivative of HT1080) cell lines and that the cellular ligands for ICAM-4 are the α₄β₁ integrin and αᵥ integrins (most notably αᵥβ₁ and αᵥβ₅) respectively (Spring et al., 2001, Blood 98: 458-466).

ICAM-4 possibly has a role in the formation of erythroblastic islands in the bone marrow (during erythropoiesis) and in the abnormal adhesion of red cells to activated endothelium in sickle cell disease. There is a need to understand interactions of ICAM-4 with its receptors for the development of therapies to diseases in which ICAM-4 is involved. These diseases include those involving pathology resulting from abnormal adhesion of red cells to vascular endothelium either directly, or indirectly through binding to other adhesive cells or molecules. Abnormal red cell adhesion is evident in sickle cell disease and malaria. Red cells from patients with β-thalassaemia major and β-thalassaemia intermedia also show increased adherence to endothelium and it has been suggested that this contributes to the microcirculatory disorders seen in these patients. Red cell-endothelial cell adherence has also been reported to contribute to the vascular complications found in diabetes mellitus. Red cell-endothelial cell adherence and red cell adherence to other cellular elements in the blood and wider reticuloendothelial system may also be relevant to the pathophysiology of other conditions where endothelial perturbation or vascular dysfunction occurs; such as strokes, organ transplant rejection, systemic lupus erythematosus and a range of vasculitic and thrombotic disorders. There is preliminary evidence for the involvement of red cell adhesion via ICAM-4 in sickle cell disease and deep vein thrombosis.

According to the present invention, there is provided an epitope for binding integrins, comprising the A and G strands of domain 1 of ICAM-4 (SEQ ID NO: 1), in which the A strand (SEQ ID NO: 2) is defined by amino acid residues 17 to 27 of ICAM-4 and the G strand (SEQ ID NO: 3) is defined by amino acid residues 90 to 100 of ICAM-4, or a functional homologue of the epitope.

The epitope was identified using site-directed mutagenesis of residues identified using a molecular model of ICAM-4 derived from the crystal structure of ICAM-2 (see FIG. 2). The term “ICAM-4” refers herein to the mature form of the human protein (as shown in SEQ ID NO: 1), without the N-terminal signal peptide of 30 amino acids found in precursor ICAM-4 (see Bailly et al., 1994, Proc. Natl. Acad. Sci. USA91: 5306-5310). Amino acid residues are numbered with reference to this mature ICAM-4. As described in further detail below, our model predicts ICAM-4 i to have two immunoglobulin superfamily I-set domains, domain 1 being N-terminal of a membrane-anchored domain 2. According to the model, Domain 1 is an I-1 subset fold with six strands that run in order A, B, C, D, E, F and G. Hence in Domain 1 there is an ABE face and a CDFG (CFG) face. Domain 2 is an I-2 subset fold with seven strands that run in order A, B, C, C′, E, F and G. Hence in Domain 2 there is an ABE and a CC′FG face. Reference to strands herein thus cover both domain 1 or domain 2 faces.

The epitope of the invention may be defined by amino acid residues F18, W19, V20 on the A strand of ICAM-4 and amino acid residues R92, A94, T95, S96 and R97 on the G strand of ICAM-4.

The epitope of the invention may be modified in that the A strand is replaced by strand F on domain 1 of ICAM-4, in which the F strand (SEQ ID NO: 4) is defined by amino acid residues 77 to 87 of ICAM-4. The epitope here may be defined by amino acid residues W77 and L80 on the F strand of ICAM-4 and amino acid residues R92, A94, T95, S96 and R97 on the G strand of ICAM-4. In the experimental section below, it is shown integrin ligands of ICAM-4 appear to interact with the A and F strands of ICAM-4.

Mutagenesis of human ICAM-4 has revealed that modification of the above-defined single amino acids affect, for example, αᵥ integrin-mediated adhesion to ICAM-4, as elaborated in the experimental section below.

The epitope of the invention may be further defined by amino acid residues W66 on the E strand of domain 1 and K118 on the B strand of domain 2 of ICAM-4, in which the E strand (SEQ ID NO: 5) is defined by amino acid residues 160 to 170 of ICAM-4 and the B strand (SEQ ID NO: 6) is defined by amino acid residues 116 to 126 of ICAM-4.

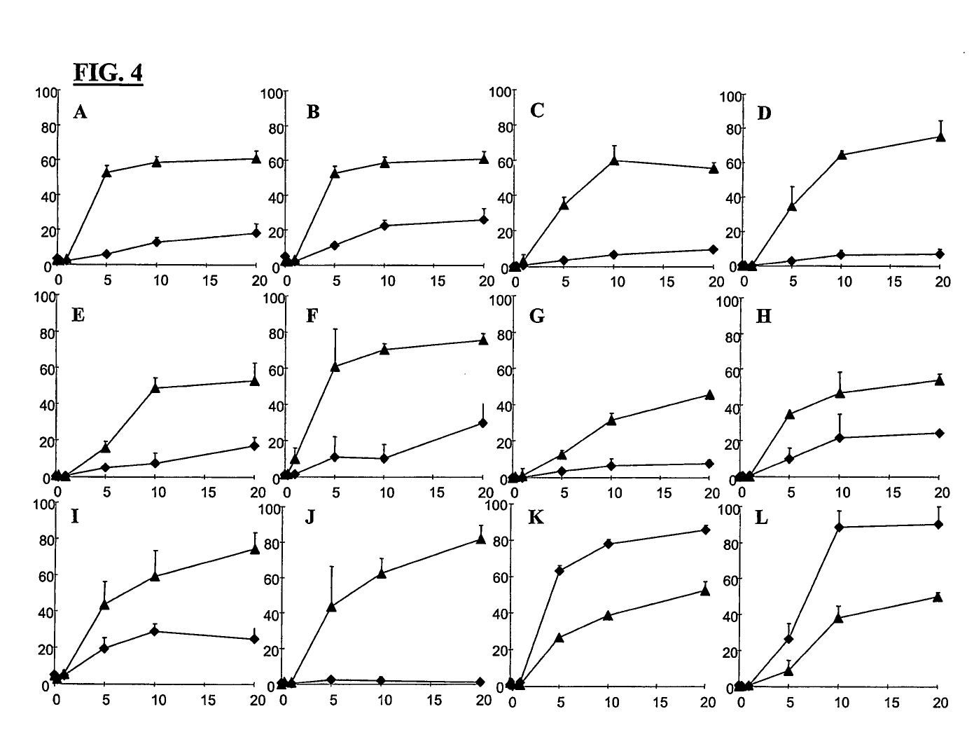

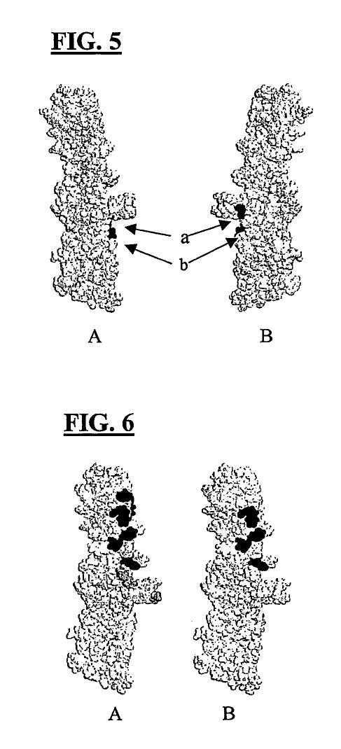

The epitope maybe further defined by amino acid residues N160, V161 and T162 on the E strand of ICAM-4. These residues define an N-glycosylation site which may have a role in the binding of ICAM-4 and its ligands. The glycosylation site is located on the top of the E strand (residues 160-170) of domain 2 (see FIG. 5). Without an N-glycan chain formed at the N-glycosylation site, the adhesion between ICAM-4 and its ligands (for example αᵥ ligand) is stronger (see FIG. 4 panels K and L, described below).

Integrins binding to the epitope or part thereof may be αᵥ integrins (for example, as found on HT1080 cells), α₄β1 (also known as VLA-4; for example, as found on HEL cells and erythroblasts), or α₅β1 (for example, as found on erythroblasts).

In another aspect of the invention, there is provided a footprint domain for binding integrins, comprising a first epitope as defined above and a second epitope comprising the C and F strands of domain 1 and the CE loop of domain 2 of ICAM-4, in which the C strand (SEQ ID NO: 7) is defined by amino acid residues 47 to 54 of ICAM-4, the F strand (SEQ ID NO: 4) is defined as above and the CE loop (SEQ ID NO: 8) is defined by amino acid residues 150 to 158 of ICAM-4, or a functional homologue of the footprint domain.

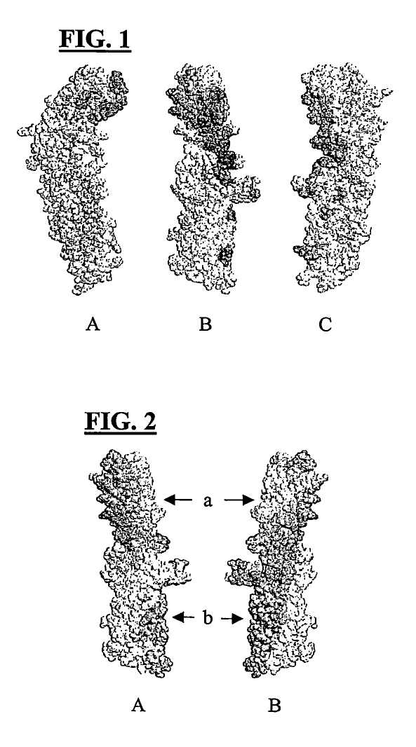

The footprint domain (depicted in FIG. 1 for ICAM-4) can be described as an “adhesive footprint” for multiple integrins. The strands of ICAM-4 as defined herein arise from their position in a molecular model of ICAM-4 that is based on the crystal structure of ICAM-2 (FIG. 2). Evidence is provided herewith for the involvement of the footprint domain in the interaction between ICAM-4 and multiple integrin ligands (see experimental section below).

The second epitope may be defined by amino acid residues R52 on the C strand of ICAM-4, W77 and L80 on the F strand of ICAM-4, T91, W93 and R97 on the G strand of ICAM-4, and E151 and T154 on the CE loop of ICAM-4. This second epitope has been disclosed by Hermand et al. (2000, J. Biol. Chem. 275: 26002-26010).

The integrins binding to the footprint domain or part thereof include αᵥ integrins (for example, as found on HT1080 cells), VLA-4 (for example, as found on HEL cells) and/or the β₂-family of integrins (such as Mac-1, for example, as found on leucocytes and neutrophils, and/or LFA-1), including αLβ2 (for example, as found on neutrophils).

Functional homologues of the epitope or footprint domain include mammalian homologues, for example mouse homologues.

Further provided according to the invention is an antagonist of the epitope and/or the footprint domain as defined herein. For example, the antagonist may be an antibody. Antibodies have the capability to directly bind to the epitope and/or footprint domain, blocking adhesion to integrin ligands. Antibodies to ICAM-4 have been described by Bailly et al. (1995, Eur. J. Immunol. 25: 3316-3320) and Goel & Diamond (2002, Blood 100: 3797-3803). It is believed that those known antibodies do not bind to the epitope or footprint domain defined herein. If this is not the case, those known antibodies are excluded from this aspect of the invention.

Alternatively, an antibody may bind a separate site on ICAM4 and alter the structural integrity of the epitope and/or footprint domain, thereby reducing affinity and/or inhibiting integrin ligand binding. It is believed that the known antibodies to ICAM described by Bailly et al. (1995, supra) and Goel & Diamond (2002, supra) do not alter the structural integrity of ICAM-4 as described above. If this is not the case, those antibodies are excluded from this aspect of the invention.

Alternatively, the antagonist of the epitope and/or the footprint domain may be a compound, for example a low molecular weight compound, which binds to the epitope and/or footprint domain to reduce adhesion between ICAM-4 and its ligands.

In another aspect of the invention there is provided an antagonist of a ligand for the epitope and/or the footprint domain defined herein. The antagonist may have or consist essentially of three, four, five, six, seven, eight, nine or more amino acid residues of the A, C, F or G strands or the CE loop of ICAM-4 or a functional homologue thereof. For example, the antagonist of a ligand for the epitope and/or the footprint domain may have or consist essentially of the amino acid sequence according to SEQ ID NO: 9, SEQ ID NO: 10 or SEQ ID NO: 11. The antagonist may comprise an active site having or consisting essentially of the amino acid sequence according to SEQ ID NO: 9, SEQ ID NO: 10 or SEQ ID NO: 11.

Experimental evidence (below) demonstrates inhibition of binding between ICAM-4 and various ligands (such as integrins).

Alternatively, the antagonist of a ligand for the epitope and/or the footprint domain may comprise other peptides, drugs or antibodies which bind to the ligand and thus reduce adhesion of the ligand to the epitope and/or the footprint domain

In a further aspect of the invention, there is provided a method of antagonising the epitope and/or the footprint domain, comprising the step of contacting the epitope and/or the footprint domain with the antagonist to the epitope and/or the footprint domain described herein. There is also provided a method of antagonising a ligand of the epitope and/or the footprint domain, comprising the step of contacting the ligand (or an environment such as a solution containing the ligand) with the antagonist of the ligand described herein. Our data shows that such antagonists (for example SEQ ID NOs: 9, 10 or 11) effective block binding to ICAM4.

Another aspect of the invention is the use of the antagonist as defined herein for treating a disease, for example a disease involving ICAM-4. Furthermore, the invention covers use of an antagonist as described herein in the manufacture of a medicament for the treatment of a disease involving ICAM-4. The disease may be characterised by increased or decreased levels of ICAM4 binding compared with ICAM-4 binding in healthy individuals.

We have found that the above epitope and footprint domain mediate adhesion to several integrins, and if this adhesion is blocked, for example, therapeutic effects may be possible in diseases such as sickle cell disease, deep vein thrombosis (DVT), malaria, strokes and more generally, vascular complications in any other condition found in mammals (heart disease, diabetes, β-thalassaemia, thrombotic complications of haematological diseases) may be possible. For example, in sickle cell disease it is thought that ICAM-4 binds sickle red cells to the endothelium. This abnormal binding may be prevented using an antagonist of ICAM-4.

In a further aspect there is provided an isolated nucleotide encoding the epitope or the footprint domain or the antagonist defined herein. For example, the isolated nucleotide encoding the epitope or the footprint domain or the antagonist may have a sequence defined within the sequence of SEQ ID NO: 12.

Embodiments of the invention will be described hereafter with reference to the accompanying figures, of which:

FIG. 1 shows a molecular model of ICAM4 depicting the entire footprint domain;

FIG. 2 shows a molecular model of ICAM-4 depicting the ABE faces and the CFG faces;

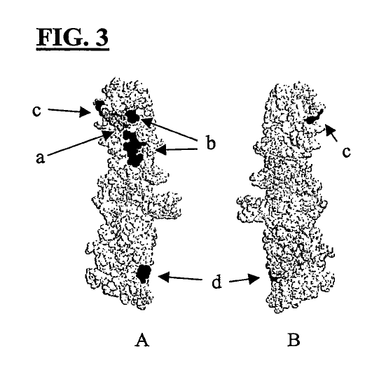

FIG. 3 shows a molecular model of the α₄β₁ and αᵥ integrin binding domain of ICAM-4;

FIG. 4 shows graphs A-L depicting the effect of mutating single residues of human ICAM-4Fc on the adhesion of HT1080 cells (exhibiting αᵥ integrin);

FIG. 5 shows a molecular model of the ICAM-4 N-glycosylation site in domain 2;

FIG. 6 shows a molecular model of the LFA-1 and Mac-i binding footprint of ICAM-4;

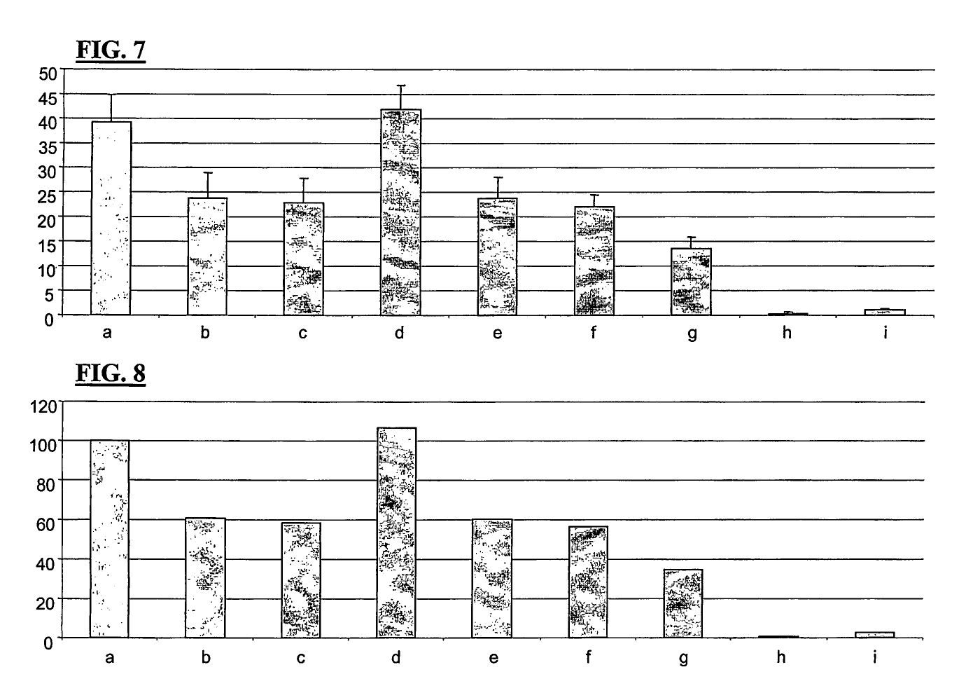

FIG. 7 is a histogram showing human ICAM-4 peptide inhibition of HEL cell binding to human ICAM-4Fc coated at 5 μg/ml;

FIG. 8 is a histogram showing the results of FIG. 7 as a percentage of binding to human ICAM-4Fc in the absence of peptides;

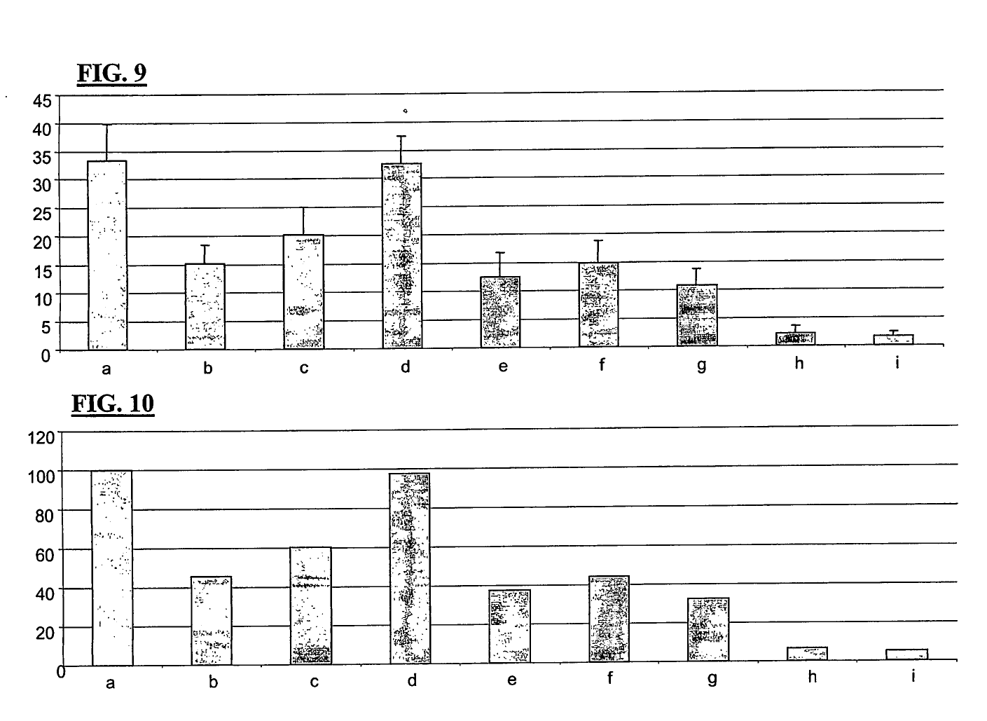

FIG. 9 is a histogram showing human ICAM-4 peptide inhibition of HT1080 cell binding to human ICAM-4Fc coated at 7.5 μg/ml;

FIG. 10 is a histogram showing the results of FIG. 8 as a percentage of binding to human ICAM-4Fc in the absence of peptides;

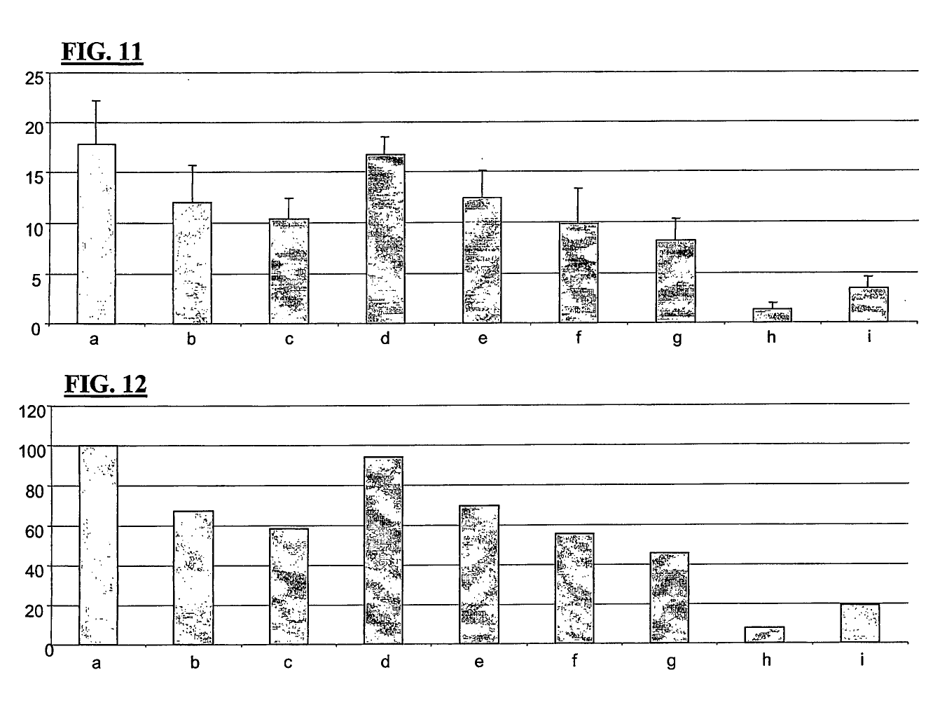

FIG. 11 is a histogram showing human ICAM-4 peptide inhibition of HEL cell binding to murine ICAM-4Fc coated at 5 μg/ml;

FIG. 12 is a histogram showing the results of FIG. 11 as a percentage of binding to murine ICAM-4Fc in the absence of peptides;

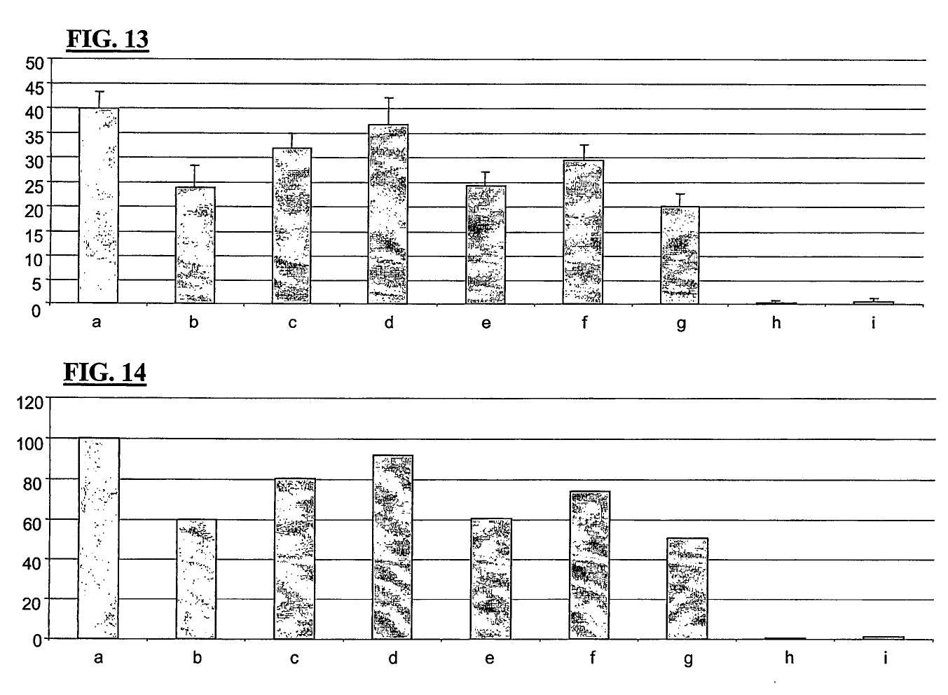

FIG. 13 is a histogram showing human ICAM-4 peptide inhibition of HT1080 cell binding to murine ICAM-4Fc coated at 51 μg/ml;

FIG. 14 is a histogram showing the results of FIG. 13 as a percentage of binding to murine ICAM-4Fc in the absence of peptides;

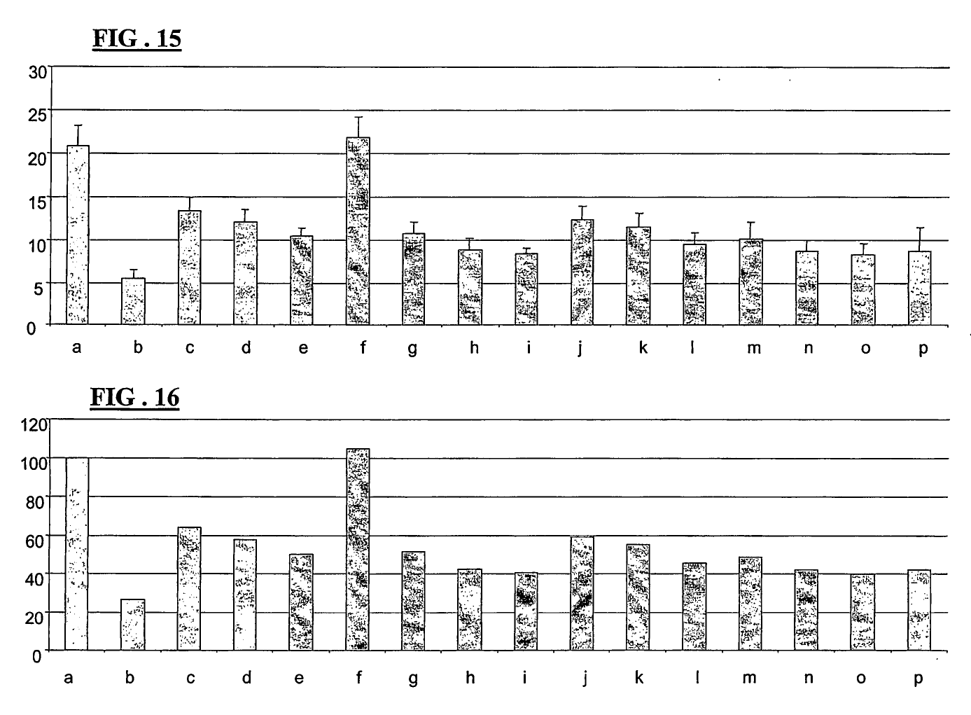

FIG. 15 is a histogram showing human ICAM-4 peptide inhibition of HEL cell binding to human ICAM-4Fc coated at 2.5 μg/ml;

FIG. 16 is a histogram showing the results of FIG. 15 as a percentage of binding to ICAM-4Fc in the absence of peptides;

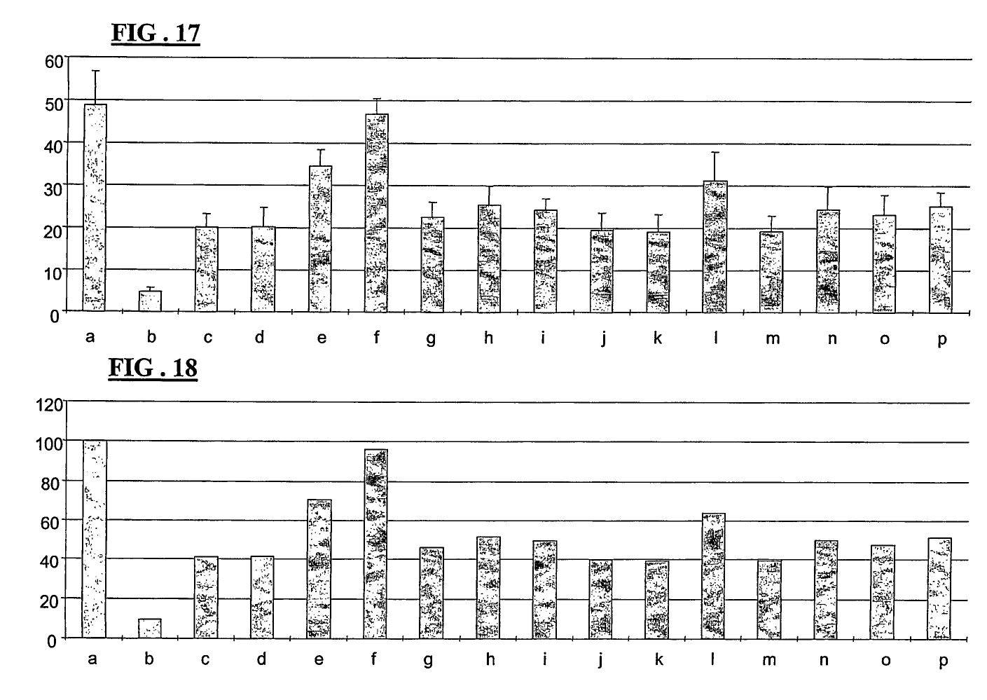

FIG. 17 is a histogram showing human ICAM-4 peptide inhibition of HT1080 cell binding to human ICAM-4Fc coated at 5 μg/ml;

FIG. 18 is a histogram showing the results of FIG. 17 as a percentage of binding to ICAM-4Fc in the absence of peptides;

FIG. 19 is a histogram showing that ICAM-4 peptides block adhesion between erythroblasts and ICAM-4; and

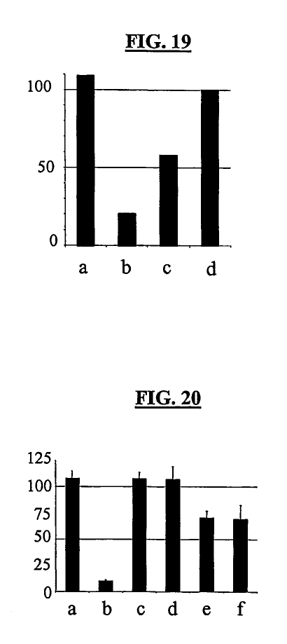

FIG. 20 is a histogram showing that blocking beta 2 antibody and ICAM-4 peptides inhibit adhesion between neutrophils and ICAM4.

The figure legends in more detail are:

FIG. 1 Molecular model of ICAM-4 with the entire “footprint” (The A, G and F strand of domain 1, extending down towards the CE loop of domain 2), along with residues W66 and K118 are shown in grey. Views A, B and C are rotated 120° with respect to each other.

FIG. 2. Molecular model of ICAM4 with the ABE faces shaded grey and the CFG faces are un-shaded. Views A and B are rotated 180° with respect to each other. Domain 1 is at the top of the model and is highlighted by a and domain 2 at the bottom of the model is highlighted by b.

FIG. 3. The α₄β₁ and αᵥ integrin binding footprint of ICAM-4. Views A and B are rotated 180° with respect to each other. The mutated residues that comprise the α₄β₁ and αᵥ integrin binding footprint in the A strand are in light grey (a), and those in the G strand are in dark grey (b). Dark grey residues in the E strand of domain 1 (W66, c) and B strand of domain 2 (K 18, d) also affect α₄β₁ and aᵥ integrin binding.

FIG. 4. The effect of mutating single residues of human ICAM-4Fc on the adhesion of HT1080 cells. x-axis: wild-type and mutant human ICAM-4Fc coating concentration (μg/ml); y-axis: percentage of input cells bound. Triangles show titrations of wild-type ICAM-4Fc and diamonds show titrations of mutant ICAM-4Fc. A, F18A mutant; B, W19A mutant; C, V20T mutant; D, R92E mutant; E, A94L mutant; F, T95V mutant; G, S96A mutant; H, R97E mutant; I, W66A mutant; J, K118E mutant; K, N160A mutant; L, T162V mutant. Results shown are representative (one of several repeat experiments). Results are shown as mean (n=3)±1 standard deviation.

FIG. 5. The ICAM-4 N-glycosylation site in domain 2. Views A and B are rotated 180° with respect to each other. Residues N160 (a) and T162 (b) are highlighted in dark grey.

FIG. 6. The LFA-1 and Mac-1 binding footprint of ICAM-4. Views A and B are rotated 180° with respect to each other. View A shows the Mac-1 footprint with domain 1 residues in the C, F and G strands highlighted in dark grey and domain 2 residues in the C′ E loop highlighted in light grey. View B shows the LFA-1 footprint with the domain 1 residues in the F and G strands highlighted in dark grey.

FIG. 7. Human ICAM-4 peptide inhibition of HEL cell binding to human ICAM-4Fc. x-axis: binding of HEL cells in the presence of assay buffer, defined peptides or EDTA; y-axis: percentage of input cells bound. a-h shows binding to human ICAM-4Fc and i shows binding to human NCAMFc. a, assay buffer; b, svpFWVrms peptide (SEQ ID NO: 9); c, tRwATSRit peptide (SEQ ID NO: 10), d, rqgktlrgp peptide (SEQ ID NO: 13); e, svpFWVrms peptide (SEQ ID NO: 9) plus rqgktlrgp peptide (SEQ ID NO: 13); f, tRwATSRit peptide (SEQ ID NO: 10) plus rqgktlrgp peptide (SEQ ID NO: 13); g, svpFWVrms peptide (SEQ ID NO: 9) plus tRwATSRit peptide (SEQ ID NO: 10); h, EDTA; i, assay buffer. Human ICAM-4Fc was coated at a concentration of 5 μg/ml, peptides were used at 500 μM final concentration for each peptide, and each data point is the mean of three independent assays.

FIG. 8. Human ICAM-4 peptide inhibition of HEL cell binding to human ICAM-4Fc. x-axis: binding of HEL cells in the presence of assay buffer, defined peptides or EDTA; y-axis: input cells bound expressed as a percentage of binding to ICAM-4Fc in the absence of peptides. a-h shows binding to human ICAM-4Fc and i shows binding to human NCAMFc. a, assay buffer (100%); b, svpFWVrms peptide (SEQ ID NO: 9) (61%); c, tRwATSRit peptide (SEQ ID NO: 10) (58%); d, rqgktlrgp peptide (SEQ ID NO: 13) (107%); e, svpFWVrms peptide (SEQ ID NO: 9) plus rqgktlrgp peptide (SEQ ID NO: 13) (60%); f, tRwATSRit peptide (SEQ ID NO: 10) plus rqgktlrgp peptide (SEQ ID NO: 13) (56%); g, svpFWVrms peptide (SEQ ID NO: 9) plus tRwATSRit peptide (SEQ ID NO: 10) (35%/0); h, EDTA (1%); i, assay buffer (3%). Human ICAM-4Fc was coated at a concentration of 5 μg/ml, peptides were used at 500 μM final concentration for each peptide, and each data point is the mean of three independent assays.

FIG. 9. Human ICAM-4 peptide inhibition of HT1080 cell binding to human ICAM-4Fc. x-axis: binding of HT1080 cells in the presence of assay buffer, defined peptides or EDTA; y-axis: percentage of input cells bound. a-h shows binding to human ICAM-4Fc and i shows binding to human NCAMFc. a, assay buffer; b, svpFWVrms peptide (SEQ ID NO: 9); c, tRwATSRit peptide (SEQ ID NO: 10), d, rqgktlrgp peptide (SEQ ID NO: 13); e, svpFWVrms peptide (SEQ ID NO: 9) plus rqgktlrgp peptide (SEQ ID NO: 13); f, tRwATSRit peptide (SEQ ID NO: 10) plus rqgktlrgp peptide (SEQ ID NO: 13); g, svpFWVrms peptide (SEQ ID NO: 9) plus tRwATSRit peptide (SEQ ID NO: 10); h, EDTA; i, assay buffer. Human ICAM-4Fc was coated at a concentration of 7.5 μg/ml, peptides were used at 500 μM final concentration for each peptide, and each data point is the mean of three independent assays.

FIG. 10. Human ICAM-4 peptide inhibition of HT1080 cell binding to human ICAM-4Fc. x-axis: binding of HT1080 cells in the presence of assay buffer, defined peptides or EDTA; y-axis: input cells bound expressed as a percentage of binding to human ICAM4Fc in the absence of peptides. a-h shows binding to human ICAM-4Fc and i shows binding to human NCAMFc. a, assay buffer; b, svpFVWVrms peptide (SEQ ID NO: 9) (46%); c, tRwATSRit peptide (SEQ ID NO: 10) (60%); d, rqgktkgp peptide (SEQ ID NO: 13) (97%); e, svpFWVrms peptide (SEQ ID NO: 9) plus rqgktlrgp peptide (SEQ ID NO: 13) (37%); f, tRwATSRit peptide (SEQ ID NO: 10) plus rqgktlrgp peptide (SEQ ID NO: 13) (44%); g, svpFWVrms peptide (SEQ ID NO: 9) plus tRwATSRit peptide (SEQ ID NO: 10) (32%); h, EDTA (6%); i, assay buffer (5%). Human ICAM-4Fc was coated at a concentration of 7.5 μg/ml, peptides were used at 500 μM final concentration for each peptide, and each data point is the mean of three independent assays.

FIG. 11. Human ICAM-4 peptide inhibition of HEL cell binding to murine ICAM-4Fc. x-axis: binding of HEL cells in the presence of assay buffer, defined peptides or EDTA; y-axis: percentage of input cells bound. a-h shows binding to murine ICAM-4Fc and i shows binding to human NCAMFc. a, assay buffer; b, svpFWVrms peptide. (SEQ ID NO: 9); c, tRwATSRit peptide (SEQ ID NO: 10), d, rqgktlrgp peptide (SEQ ID NO: 13); e, svpFWVrms peptide (SEQ ID NO: 9) plus rqgkllrgp peptide (SEQ ID NO: 13); f, tRwATSRit peptide (SEQ ID NO: 10) plus rqgktlrgp peptide (SEQ ID NO: 13); g, svpFWVrms peptide (SEQ ID NO: 9) plus tRwATSRit peptide (SEQ ID NO: 10); h, EDTA; i, assay buffer. Murine ICAM-4Fc was coated at a concentration of 5 μg/ml, peptides were used at 500 μM final concentration for each peptide, and each data point is the mean of two independent assays.

FIG. 12. Human ICAM-4 peptide inhibition of HEL cell binding to murine ICAM-4Fc. x-axis: binding of HEL cells in the presence of assay buffer, defined peptides or EDTA; y-axis: input cells bound expressed as a percentage of binding to murine ICAM-4Fc in the absence of peptides. a-h shows binding to murine ICAM-4Fc and i shows binding to human NCAMFc. a, assay buffer; b, svpFWVrms peptide (SEQ ID NO: 9) (67%); c, tRwATSRit peptide (SEQ ID NO: 10) (58%); d, rqgktlrgp peptide (SEQ ID NO: 13) (94%); e, svpFWVrms peptide (SEQ ID NO: 9) plus rqgktlrgp peptide (SEQ ID NO: 13) (70%); f, tRwATSRit peptide (SEQ ID NO: 10) plus rqgktlrgp peptide (SEQ ID NO: 13) (55%); g, svpFWVrms peptide (SEQ ID NO: 9) plus tRwATSRit peptide (SEQ ID NO: 10) (47%); h, EDTA (8%); i, assay buffer (19%). Murine ICAM-4Fc was coated at a concentration of 5 μg/ml, peptides were used at 500 μM final concentration for each peptide, and each data point is the mean of two independent assays.

FIG. 13. Human ICAM-4 peptide inhibition of HT1080 cell binding to murine ICAM-4Fc. x-axis: binding of HT1080 cells in the presence of assay buffer, defined peptides or EDTA; y-axis: percentage of input cells bound. a-h shows binding to murine ICAM-4Fc and i shows binding to human NCAMFc. a, assay buffer; b, svpFWVrms peptide (SEQ ID NO: 9); c, tRwATSRit peptide (SEQ ID NO: 10), d, rqgktlrgp peptide (SEQ ID NO: 13); e, svpFWVrms peptide (SEQ ID NO: 9) plus rqgktlrgp peptide (SEQ ID NO: 13); f, tRwATSRit peptide (SEQ ID NO: 10) plus rqgktlrgp peptide (SEQ ID NO: 13); g, svpFWVrms peptide (SEQ ID NO: 9) plus tRwATSRit peptide (SEQ ID NO: 10); h, EDTA; i, assay buffer. Murine ICAM-4Fc was coated at a concentration of 5 μg/ml, peptides were used at 500 μM final concentration for each peptide, and each data point is the mean of two independent assays.

FIG. 14. Human ICAM-4 peptide inhibition of HT1080 cell binding to murine ICAM-4Fc. x-axis: binding of HT1080 cells in the presence of assay buffer, defined peptides or EDTA; y-axis: input cells bound expressed as a percentage of binding to murine ICAM-4Fc in the absence of peptides. a-h shows binding to murine ICAM-4Fc and i shows binding to human NCAMFc. a, assay buffer; b, svpFWVrms peptide (SEQ ID NO: 9) (60%); c, tRwATSRit peptide (SEQ ID NO: 10) (80%); d, rqgktlrgp peptide (SEQ ID NO: 13) (92%); e, svpFWVrms peptide (SEQ ID NO: 9) plus rqgktlrgp peptide (SEQ ID NO: 13) (61%); f, tRwATSRit peptide (SEQ ID NO: 10) plus rqgktlrgp peptide (SEQ ID NO: 13) (74%); g, svpFWVrms peptide (SEQ ID NO: 9) plus tRwATSRit peptide (SEQ ID NO: 10) (51%); h, EDTA (1%); i, assay buffer (1%). Murine ICAM-4Fc was coated at a concentration of 5 μg/ml, peptides were used at 500 μM final concentration for each peptide, and each data point is the mean of two independent assays.

FIG. 15. Human ICAM-4 peptide inhibitions of HEL cell binding to human ICAM-4Fc. x-axis: binding of HEL cells in the presence of assay buffer, defined peptides or EDTA, y-axis: percentage of input cells bound. a-p shows binding to human ICAM-4Fc. a, assay buffer, b, assay buffer plus 2 mM EDTA c svpFWVrms peptide (SEQ ID NO: 9), d, tRwATSRit peptide (SEQ ID NO: 10), e, aWssLahcl peptide (SEQ ID NO: 11), f, rqgktlrgp peptide (SEQ ID NO: 13), g, svpFWVrms peptide (SEQ ID NO: 9) plus tRwATSRit peptide (SEQ ID NO: 10), h, svpFWVrms peptide (SEQ ID NO: 9) plus aWssLahcl peptide (SEQ ID NO: 11), i, tRwATSRit peptide (SEQ ID NO: 10) plus aWssLahcl peptide (SEQ ID NO: 11), j, svpFWVrms peptide (SEQ ID NO: 9) plus rqgktlrgp peptide (SEQ ID NO: 13), k, tRwATSRit peptide (SEQ ID NO: 10) plus rqgktlrgp peptide (SEQ ID NO: 13), 1, aWssLahcl peptide (SEQ ID NO: 11) plus rqgktlrgp peptide (SEQ ID NO: 13), m, svpFWVrms peptide (SEQ ID NO: 9) plus tRwATSRit peptide (SEQ ID NO: 10) plus rqgktlrg peptide (SEQ ID NO: 13), n, svpFWVrms peptide (SEQ ID NO: 9) plus aWssLahcl peptide (SEQ ID NO: 11) plus rqgktlrgp peptide (SEQ ID NO: 13), o, tRwATSRit peptide (SEQ ID NO: 10) plus aWssLahcl peptide (SEQ ID NO: 11) plus rqgktlrgp peptide (SEQ ID NO: 13), p, svpFWVrms peptide (SEQ ID NO: 9) plus tRwATSRit peptide (SEQ ID NO: 10) plus aWssLahcl peptide (SEQ ID NO: 11). Human ICAM-4Fc was coated at a concentration of 2.5 μg/ml, peptides were used at 750 μM final concentration for each peptide, and each data point is the mean of two independent assays

FIG. 16. Human ICAM-4 peptide inhibitions of HEL cell binding to human ICAM-4Fc. x-axis: binding of HEL cells in the presence of assay buffer, defined peptides or EDTA, y-axis: input cells bound expressed as a percentage of binding to human ICAM-4Fc in the absence of peptides. a, assay buffer; b, assay buffer plus 2 mM EDTA (26%); c, svpFWVrms peptide (SEQ ID NO: 9) (64%); d, tRwATSRit peptide (SEQ ID NO: 10) (58%); e, aWssLahcl peptide (SEQ ID NO: 11) (50%/o); f, rqgktlrgp peptide (SEQ ID NO: 13) (105%); g, svpFWVrms peptide (SEQ ID NO: 9) plus tRwATSRit peptide (SEQ ID NO: 10) (52%); h, svpFWVrms peptide (SEQ ID NO: 9) plus aWssLahcl peptide (SEQ ID NO: 11) (43%); i, tRwATSRit peptide (SEQ ID NO: 10) plus aWssLahcl peptide (SEQ ID NO: 11) (41%); j, svpFWVrms peptide (SEQ ID NO: 9) plus rqgktlrgp peptide (SEQ ID NO: 13) (59%); k, tRwATSRit peptide (SEQ ID NO: 10) plus rqgktlrgp peptide (SEQ ID NO: 13) (55%); 1, aWssLahcl peptide (SEQ ID NO: 11) plus rqgktlrgp peptide (SEQ ID NO: 13) (46%); m, svpFWVrms peptide (SEQ ID NO: 9) plus tRwATSRit peptide (SEQ ID NO: 10) plus rqgktlrg peptide (49%/o); n, svpFWVrms peptide (SEQ ID NO: 9) plus aWssLahcl peptide (SEQ ID NO: 11) plus rqgktlrgp peptide (SEQ ID NO: 13) (42%); o, tRwATSRit peptide (SEQ ID NO: 10) plus aWssLahcl peptide (SEQ ID NO: 11) plus rqgktlrgp peptide (SEQ ID NO: 13) (40%); p, svpFWVrms peptide (SEQ ID NO: 9) plus tRwATSRit peptide (SEQ ID NO: 10) plus aWssLahcl peptide (SEQ ID NO: 11) (42%). Human ICAM-4Fc was coated at a concentration of 2.5 μg/ml, peptides were used at 750 μM final concentration for each peptide, and each data point is the mean of two independent assays.

FIG. 17. Human ICAM-4 peptide inhibitions of HT1080 cell binding to human ICAM-4Fc. x-axis: binding of HT1080 cells in the presence of assay buffer, defined peptides or EDTA, y-axis: percentage of input cells bound. a-p shows binding to human ICAM-4Fc. a, assay buffer, b, assay buffer plus 2 mM EDTA c svpFWVrms peptide (SEQ ID NO: 9), d, tRwATSRit peptide (SEQ ID NO: 10), e, aWssLahcl peptide (SEQ ID NO: 11), f, rqgktlrgp peptide (SEQ ID NO: 13), g, svpFWVrms peptide (SEQ ID NO: 9) plus tRwATSRit peptide (SEQ ID NO: 10), h, svpFWVrms peptide (SEQ ID NO: 9) plus aWssLahcl peptide (SEQ ID NO: 11), i, tRwATSRit peptide (SEQ ID NO: 10) plus aWssLahcl peptide (SEQ ID NO: 11), j, svpFWVrms peptide (SEQ ID NO: 9) plus rqgktlrgp peptide (SEQ ID NO: 13), k, tRwATSRit peptide (SEQ ID NO: 10) plus rqgktlrgp peptide (SEQ ID NO: 13), 1, aWssLahcl peptide (SEQ ID NO: 11) plus rqgktlrgp peptide (SEQ ID NO: 13), m, svpFWVrms peptide (SEQ ID NO: 9) plus tRwATSRit peptide (SEQ ID NO: 10) plus rqgktlrg peptide, n, svpFWVrms peptide (SEQ ID NO: 9) plus aWssLahcl peptide (SEQ ID NO: 11) plus rqgktlrgp peptide (SEQ ID NO: 13), o, tRwATSRit peptide (SEQ ID NO: 10) plus aWssLahcl peptide (SEQ ID NO: 11) plus rqgktlrgp peptide (SEQ ID NO: 13), p, svpFWVrms peptide (SEQ ID NO: 9) plus tRwATSRit peptide (SEQ ID NO: 10) plus aWssLahcl peptide (SEQ ID NO: 11). Human ICAM-4Fc was coated at a concentration of 5 μg/ml, peptides were used at 750 μM final concentration for each peptide, and each data point is the mean of two independent assays.

FIG. 18. Human ICAM-4 peptide inhibitions of HT1080 cell binding to human ICAM-4Fc. x-axis: binding of HT1080 cells in the presence of assay buffer, defined peptides or EDTA, y-axis: input cells bound expressed as a percentage of binding to human ICAM-4Fc in the absence of peptides. a, assay buffer; b, assay buffer plus 2 mM EDTA (10%); c, svpFWVrms peptide (SEQ ID NO: 9) (41%); d, tRwATSRit peptide (SEQ ID NO: 10) (42%); e, aWssLahcl peptide (SEQ ID NO: 11) (71%); f, rqgktlrgp peptide (SEQ ID NO: 13) (96%); g, svpFWVrVns peptide (SEQ ID NO: 9) plus tRwATSRit peptide (SEQ ID NO: 10) (46%); h, svpFWVnns peptide (SEQ ID NO: 9) plus aWssLahcl peptide (SEQ ID NO: 11) (52%); i, tRwATSRit peptide (SEQ ID NO: 10) plus aWssLahcl peptide (SEQ ID NO: 11) (50%); j, svpFWVrms peptide (SEQ ID NO: 9) plus rqgktlrgp peptide (SEQ ID NO: 13) (40%/o); k, tRwATSRit peptide (SEQ ID NO: 10) plus rqgktlrgp peptide (SEQ ID NO: 13) (39%); 1, aWssLahcl peptide (SEQ ID NO: 11) plus rqgktlrgp peptide (SEQ ID NO: 13) (64%); m, svpFWVrms peptide (SEQ ID NO: 9) plus tRwATSRit peptide (SEQ ID NO: 10) plus rqgktlrg peptide (39%); n, svpFWVrms peptide (SEQ ID NO: 9) plus aWssLahcl peptide (SEQ ID NO: 11) plus rqgktlrgp peptide (SEQ ID NO: 13) (50%); o, tRwATSRit peptide (SEQ ID NO: 10) plus aWssLahcl peptide (SEQ ID NO: 11) plus rqgktlrgp peptide (SEQ ID NO: 13) (48%); p, svpFWVrms peptide (SEQ ID NO: 9) plus tRwATSRit peptide (SEQ ID NO: 10) plus aWssLahcl peptide (SEQ ID NO: 11) (52%). Human ICAM-4Fc was coated at a concentration of 5 μg/ml, peptides were used at 750 μM final concentration for each peptide, and each data point is the mean of two independent assays.

FIG. 19. ICAM-4 peptides block adhesion between erythroblasts and ICAM-4. Erythroblast adhesion to 5 μg/ml ICAM-4 was performed in the presence of 5 μg/ml of the beta 1 integrin activating antibody TS2/16. Peptides were used at 500 μM. Results are expressed as a % of the erythroblasts bound to ICAM4 minus those bound to NCAM in the presence of the rqgktlrgp peptide (SEQ ID NO: 13) (i.e. % control cells bound). a, svpFWVrms peptide (SEQ ID NO: 9); b, tRwATSRit peptide (SEQ ID NO: 10); c, aWssLahcl peptide (SEQ ID NO: 11); d, rqgktlrgp peptide (SEQ ID NO: 13).

FIG. 20. Blocking beta 2 antibody and ICAM-4 peptides inhibit adhesion between neutrophils and ICAM-4. Neutrophil adhesion to 2.5 μg/ml ICAM-4 was performed in the presence of 10 μg/ml of the integrin blocking antibodies and 500 μM of the peptides. Results are expressed as a % of the neutrophils bound to ICAM-4 in the absence of either antibody or peptide (i.e. % control cells bound). a, beta 1; b, beta 2; c, beta 3; d, svpFWVrms peptide (SEQ ID NO: 9); e, tRwATSRit peptide (SEQ ID NO: 10); f, aWssLahcl peptide (SEQ ID NO: 11).

### Experimental

In order to elucidate the structural basis of integrin-ICAM-4 interaction, in Example 1 we analysed surface-exposed residues, by site-directed mutagenesis, using a molecular model of ICAM-4 derived from the crystal structure of ICAM-2. The model presents ICAM-4 as two Ig-like domains; domain 1 being N-terminal of the membrane anchored domain 2. Each domain has two faces (or sides); the ABE and the CC′FG faces (FIG. 2). Mutagenesis of ICAM-4 has revealed that a number of single amino acid changes affect αᵥ integrin-mediated adhesion to ICAM-4. Peptide inhibition data confirms the mutagenesis data and provides evidence that the same footprint is relevant to ICAM-4's interaction with α₄β₁. Due to the overlap of the αᵥ integrin and a4p, binding site with that of the binding site of LFA-1 and Mac-1 we predict that the peptides also inhibit any ICAM4/LFA-1 or Mac-1 interaction. In Examples 2 and 3, we show that blocking peptides (antagonists) are capable of inhibiting erythroblast and neutrophil adhesion to ICAM-4, suggesting that such antagonists can be useful in treating diseases relating to ICAM-4 dysfunction.

### EXAMPLE 1

Cell Adhesion Assay

Cell adhesion assays were performed as described in Spring et al. 2001 (supra). Immulon4 96 well plates (Dynex Technologies, Billingshurst, United Kingdom) were coated with 1 μg/well goat-antihuman-Fc (Sigma, Poole, United Kingdom) for 24 hours at 4° C., washed three times with PBS and coated with an Fc fusion ICAM-4 protein for 18 hours at 4° C. before blocking with 0.4% BSA PBS for 2 hours at 22° C. Cells were labelled with 10 μg/ml 2′,7′-bis-(2-carboxyethyl)-5-(and-6-) carboxyfluorescein acetoxymethyl ester in assay buffer (IMEM, 2 mM EGTA, 10 μg/ml human ivIgG) for 15 minutes at 37° C. HT1080 cells were activated with 80 μM phorbol myristate acetate prior to both cells being washed with assay buffer containing 2 mM Mn²⁺. Cells, at 5×10⁴ cells per well, were added to the ICAM-4Fc-coated plates for 30 minutes at 37° C., prior to being given repeated washes in assay buffer and read on a fluorescence microplate reader (excitation 485 nm, emission 530 nm). The percentage of bound cells was calculated after each wash. Peptide inhibition was performed by incubating the cells with 500 μM peptide at 0° C. for 15 minutes ahead of their addition, still in the presence of 500 μM peptide to the ICAM-4Fc coated plates. In peptide inhibition studies the appropriate ICAM-4Fc coating concentration for each cell line was pre-determined by titration of ICAM-4Fc and the lowest concentration at which maximal binding was achieved was used to coat the plates

Preparation of ICAM-4Fc Fusion Proteins

Point mutations were inserted into ICAM-4 in pIg vector (see Simmons D L, 1993, Cloning cell surface molecules by transient expression in mammalian cells, In: Hartley D A, ed. Cellular interactions in development. New York, N.Y.:IRL press, 93-127) by PCR amplification over two stages. Oligonucleotides (see “Mutagenesis primers” below) containing mismatched bases, together with 5′-agaacccactgcttactggct (SEQ ID NO: 14) and 3′-tgagcctgcttccagcagca (SEQ ID NO: 15) primers were used to generate two overlapping products. Following gel purification the two overlapping PCR products were annealed together before final amplification using the 5′ and 3′ primers. The final PCR product was restricted and ligated into pIg vector. All mutant clones were verified by sequence analysis. Mutant ICAM-4Fc proteins were expressed in COS-7 cells as described in Simmons D L (1993, supra), and purified from culture supernatant on protein A-Sepharose.

TABLE 1

Mutagenesis primers
(all shown in 5′-3′ orientation)

| | | |
|---|---|---|
| F18A | tca gtg ccc GCc tgg gtg cgc | (SEQ ID NO:16) |
| | gcg cac cca gGC ggg cac tga | (SEQ ID NO:17) |
| W19A | gtg ccc ttc GCg gtg cgc atg | (SEQ ID NO:18) |
| | cat gcg cac cGC gaa ggg cac | (SEQ ID NO:19) |
| V20T | ccc ttc tgg ACg cgc atg agc | (SEQ ID NO:20) |
| | gct cat gcg cGT cca gaa ggg | (SEQ ID NO:21) |
| R92E | gga aaa aca GAA tgg gcc ac | (SEQ ID NO:22) |
| | gt ggc cca TTC tgt ttt tcc | (SEQ ID NO:23) |
| A94L | aca cgc tgg CTc acc tcc agg | (SEQ ID NO:24) |
| | cct gga ggt gAG cca gcg tgt | (SEQ ID NO:25) |
| T95V | cgc tgg gcc GTc tcc agg at | (SEQ ID NO:26) |
| | at cct gga gAC ggc cca gcg | (SEQ ID NO:27) |
| S96A | tgg gcc acc Gcc agg atc acc | (SEQ ID NO:28) |
| | ggt gat cct ggC ggt ggc cca | (SEQ ID NO:29) |
| R97E | gcc acc tcc GAg atc acc gc | (SEQ ID NO:30) |
| | gc ggt gat cTG gga ggt ggc | (SEQ ID NO:31) |
| W66A | ggg ccg ggt GCg gtg tct ta | (SEQ ID NO:32) |
| | ta aga cac cGC acc cgg ccc | (SEQ ID NO:33) |
| K118E | aag ggc agg Gaa tac act tt | (SEQ ID NO:34) |
| | aa agt gta ttC cct gcc ctt | (SEQ ID NO:35) |
| N160A | gat ctg gcc GCc gtg acc ttg | (SEQ ID NO:36) |
| | caa ggt cac gGC ggc cag atc | (SEQ ID NO:37) |
| T162V | gcc aac gtg GTc ttg acc ta | (SEQ ID NO:38) |
| | ta ggt caa gAC cac gtt ggc | (SEQ ID NO:39) |

Results and Discussion

ICAM-4 is predicted to have two immunoglobulin superfamily I-set domains, domain 1 being N-terminal of the membrane anchored domain 2. On a molecular model of ICAM-4 (Spring et al. 2001, supra, and see FIG. 2), based on the crystal structure of ICAM-2, the VLA-4 and αᵥ integrin binding epitope on ICAM-4 consists of eight residues and is located in domain 1, in between the ABE and CFG faces (FIG. 3). Three of the residues, F18, W19, and V20, are positioned on the A strand (FIG. 3a), and five residues, R92, A94, T95, S96 and R97 are on the G strand (FIG. 3b). Each of these residues was identified as important for binding on the basis of a decrease in binding of the singly mutated ICAM-4 and the αᵥ integrin ligand (see FIG. 4 panels A through L). These residues identify an epitope on the ICAM-4 molecule that straddles the edges of both the ABE and CC′FG face of domain 1.

There is also a published LFA-1/Mac-1 binding site (Hermand et al., 2000, supra) on ICAM-4 which is comprised of 8 residues, T91, R52, E151, T154, W93, L80, R97 and W77. On domain 1, T91, W93 and R97 are on the G strand (residues 90-100), W77 and L80 are on the F strand (residues 77-87) and R52 is on the C strand (residues 47-54). On domain 2 E151 and T154 are on the C′-E loop (residues 150-158). Of these residues, all comprise the Mac-1 binding site (FIG. 6 view A) however, W93, L80, R97 and W77 only comprise the LFA-1 binding site (FIG. 6 view B).

In total, the footprint domain of the present invention comprises a wider area than that covered by the epitope defined by the residues mutated herein (see FIG. 4). It comprises these residues, the residues described by Hermand et al. (2000, supra) and amino-acids in the surrounding area. The footprint comprises residues on the A, C, G and F strand of domain 1 and extends down to the CE loop in domain 2 (see FIGS. 1, 2, 3, 5, and 6).

Two other residues are thought to be involved in the interaction between ICAM-4 and its integrin ligands; W66, located on the E strand (residues 65-75) of domain 1 and K118, which is found on the B strand (residues 116-126) of domain 2 (FIG. 3 view A and residues c and d, respectively and FIG. 4 panels I and J respectively). These mutations also decrease the level of adhesion between ICAM-4 and HT1080 cells.

In addition, an N-glycosylation site comprising residues N160, V161 and T162 is believed to have a role in the binding of ICAM-4 and its ligands. This site is located at the top of the E strand (residues 160-170) of domain 2 (see FIG. 5 views A and B; N160 is arrowed by a, and T162 is arrowed by b). Mutation of N160 or T162 leads to an elevated level of adhesion between ICAM-4 and HT1080 cells (FIG. 4 panels K and L). Analysis by sodium dodecyl sulphate-polyacrylamide gel electrophoresis revealed that the N160A and T162V mutants have increased electrophoretic mobility than native ICAM-4, which suggests that these two “super adhesive” mutations prevent the N-glycosylation of asparagine 160.

Areas thought to be important in ICAM-4 binding are shown in FIGS. 1, 3, 5 and 6.

FIGS. 7-18 show inhibition of HEL cell binding and HT1080 cell binding to human and murine ICAM-4Fc in the presence of blocking peptide sequences (peptides svpFWVrms (SEQ ID NO: 9), tRwATSRit (SEQ ID NO: 10) and aWssLahcl (SEQ ID NO: 11)) and a control peptide (rqgktlrgp; SEQ ID NO: 13).

Our findings suggest that contact between ICAM-4 and its integrin ligands involves a large extent of the surface of ICAM-4, with the epitope on domain 1 being a critical site in mediating this interaction. Integrin-mediated adhesion to ICAM-4 may play a role in the formation of erythroblastic islands in the bone marrow (during erythropoiesis) and in the abnormal adhesion of red cells to activated endothelium and other cellular elements in the vasculature and wider reticuloendothelial system in the diseases mention above.

### EXAMPLE 2

Peptide Inhibition of Erythroblast Adhesion to ICAM-4

In Example 1, we identified an area on ICAM-4 that is important in its adhesion to αV integrins and using this information we designed blocking peptides corresponding to the sequences of the A, D, F and G strands of domain 1. These peptides have the sequences S(15)VPFWVRMS (SEQ ID NO: 9; on A strand), R(56)QGKTLRGP (SEQ ID NO: 13; on D strand), A(76)WSSLAHCL (SEQ ID NO: 11; on F Strand) and T(91)RWATSRIT (SEQ ID NO: 10; on G strand). We have shown that early erythroblasts bind to ICAM-4 in the presence of TS2/16, an activating β1 antibody (unpublished observations). The adhesion of HEL cells to ICAM-4 is mediated by the α4β1 integrin but not the α5β1 integrin (Spring et al., 2001, supra). Erythroblasts express only two integrins at this stage in differentiation: α4β1 and α5β1 (unpublished observations). Therefore we hypothesise that erythroblasts adhere to ICAM-4 via α4β1, although we have not ruled out the fact that α5β1 may be involved in this interaction.

We have utilised the blocking ICAM-4 peptides (i.e., SEQ ID NOs: 9, 10, 11 and 13—see Example 1) in order to inhibit the adhesion of day 4 erythroblasts to ICAM-4 (see FIG. 19). Erythroblast cultures were initiated from CD34 positive cells purified from pooled buffy coat residues (obtained from the National Blood Service, Bristol, UK) and maintained as described in Southcott et al. (1999, Blood 93: 4425-4435). Cell adhesion assays were performed as described in Example 1 above. Immulon-4 96 well plates (Dynes Technologies, Billingshurst, UK) were coated with 1 μg/well goat-antihuman-Fc (Sigma, Poole, UK) for 24 hours at 4° C., washed three times with PBS and coated with 0.25 μg/well Fc fusion ICAM-4 protein (ICAM-4Fc) for 18 hours at 4° C. before blocking with 0.4% BSA PBS for 2 hours at 22° C. Erythroblasts were labelled with 10 μg/ml 2′,7′-bis-(2-carboxyethyl)-5-(and-6-)carboxyfluorescein acetoxymethyl ester (Sigma, Poole, UK) in assay buffer (Iscoves modified Eagle medium, 2 mM EGTA, 0.1% BSA, 10 μg/ml Immune globulin intravenous (human) (Cutter Biological, Newbury, Berks, UK)) for 15 minutes at 37° C. Erythroblasts were washed with assay buffer containing 2 mM Mn²⁺. Erythroblasts, at 5×10⁴ cells per well, were added to the ICAM-4Fc-coated plates for 30 minutes at 37° C., prior to being cyclically read on a fluorescence microplate reader (excitation 485 nm, emission 530 nm) and washed in assay buffer. The percentage of bound cells was calculated after each wash Peptide inhibition was performed by incubating the cells with 500 μM peptide and 5 μg/ml TS2/16 (beta 1 activating antibody (IBGRL) at 0° C. for 15 minutes before their addition to the ICAM-4Fc coated plates.

The F and the G strand peptides (SEQ ID NOs: 10 and 11, respectively) inhibit adhesion whereas the strand A and D (SEQ ID NOs: 9 and 13, respectively) peptides had no effect. This suggests, along with the data already provided of the peptide inhibition of HEL cell—ICAM-4 adhesion (see Example 1), that the area of interaction with α4β1 on ICAM-4 lies in the F and G strands of domain 1. Therefore, the peptides of SEQ ID NOs: 9, 10 and 11 are useful tools allowing blocking of further ICAM-4 integrin interactions that are important in erythropoiesis and in the pathology of sickle cell disease, for example.

### EXAMPLE 3

Peptide and Antibody Inhibition of Neutrophil Adhesion to ICAM-4

ICAM-4 binds to platelet αIIbβ3 and the β2 integrins. These interactions may be part of the process whereby red cells participate in normal hemostatic processes and may also be relevant to thrombotic conditions such as deep vein thrombosis and vaso-occlusion in sickle cell disease. Indeed, it has recently been shown that during sickle cell crisis neutrophils that express β2 integrins, αLβ2 and αMβ2, bind not only inflamed endothelium but also adhere to erythrocytes. Since ICAM-4 is a likely, perhaps the only, candidate for mediating this erythrocyte adhesion with β2 integrins, we have assayed the in vitro adhesion of neutrophils to ICAM-4.

Utilising blocking β integrin subunit antibodies and our blocking ICAM-4 peptides (i.e., SEQ ID NOs: 9, 10, 11 and 13—see Example 1) in a microplate neutrophil adhesion assay. Neutrophils were purified from buffy coats (obtained from the National Blood Service, Bristol, UK) as described in Henderson et al. (1987, Biochem. J. 246: 325-329). Cell adhesion assays were performed as described in Example 1 above. Immulon-4 96 well plates (Dynes Technologies, Billingshurst, UK) were coated with 1 μg/well protein A (Sigma, Poole, UK) for 24 hours at 4° C., washed three times with PBS and coated with 0.125 μg/well Fc fusion ICAM-4 protein (ICAM-4Fc) for 18 hours at 4° C. before blocking with 0.4% BSA PBS for 2 hours at 22° C. Neutrophils were labelled with 10 μg/ml 2′,7′-bis-(2-carboxyethyl)-5-(and-6-)carboxyfluorescein acetoxymethyl ester (Sigma, Poole, UK) in assay buffer (Iscoves modified Eagle medium, 2 mM EGTA, 0.1% BSA for 15 minutes at 37° C. Neutrophils were washed with assay buffer containing 2 mM Mn²⁺. Neutrophils, at 5×10⁵ cells per well, were added to the ICAM-4Fc-coated plates for 10 minutes at 37° C., prior to being cyclically read on a fluorescence microplate reader (excitation 485 nm, emission 530 nm) and washed in assay buffer. The percentage of bound cells was calculated after each wash. Peptide and antibody inhibition was performed by incubating the cells with 500 μM peptide and 25 μg/ml antibody at 0° C. for 15 minutes before their addition to the ICAM-4 Fc coated plates. Antibodies used were β₁ Mab13 (Yamada UK), β₂ TS1/18 (EBGRL) and β₃ PM6/13 (Serotec, UK).

We show in FIG. 20 that neutrophil—ICAM-4 adhesion is mediated by β2 integrins (αLβ2 and αMβ2) and that it is likely to involve an interaction with the G and F strands of domain 1 of ICAM-4 as opposed to the A strand. These results are consistent with a previous site directed mutagenesis study of ICAM-4 that identified 8 residues T91, R52, E151, T154, W93, L80, R97 and W77 as important for adhesion to the β2 integrins (Hermand et al., 2000, supra). All of these residues are involved in binding αMβ2 but only W93, L80, R97 and W77 comprise the αLβ2 binding site. T91, W93, L80, R97 and W77 are all located on the G and F strands of domain 1 of ICAM-4.

Neutrophils bind the endothelium and to sickle red cells and thus are likely to be important in the blockage of capillaries (vaso-occlusion) in sickle cell disease. Example 3 shows that the adhesion between neutrophils and ICAM-4 is β2 integrin mediated and that the peptides of SEQ ID NO: 10 and 11 inhibit this interaction. This means that antagonists to ICAM-4 such as SEQ ID NO: 10 and 11 could be used to affect (for example, inhibit) hemostatic processes as well as thrombotic conditions such as deep vein thrombosis and vaso-occlusion in sickle cell disease.

1. An epitope for binding integrins, comprising strands A and G of domain 1 of ICAM4 (SEQ ID NO: 1), in which the A strand (SEQ ID NO: 2) is defined by amino acid residues 17 to 27 of ICAM-4 and the G strand (SEQ ID NO: 3) is defined by amino acid residues 90 to 100 of ICAM-4, or a functional homologue of the epitope.

2. The epitope according to claim 1, defined by amino acid residues F18, W19, V20 on the A strand of ICAM-4 and amino acid residues R92, A94, T95, S96 and R97 on the G strand of ICAM-4.

3. The epitope according to claim 1, modified in that the A strand is replaced by strand F on domain 1 of ICAM-4, in which the F strand (SEQ ID NO: 4) is defined by amino acid residues 77 to 87 of ICAM-4.

4. The epitope according to claim 3, defined by amino acid residues W77 and L80 on the F strand of ICAM-4 and amino acid residues R92, A94, T95, S96 and R97 on the G strand of ICAM-4.

5. The epitope according to claim 1, further defined by amino acid residues W66 on the E strand of domain 1 of ICAM-4 and K118 on the B strand of domain 2 of ICAM-4, in which the E strand (SEQ ID NO: 5) is defined by amino acid residues 160 to 170 of ICAM-4 and the B strand (SEQ ID NO: 6) is defined by amino acid residues 116 to 126 of ICAM-4.

6. The epitope according to claim 1, further defined by amino acid residues N160, V161 and T162 on the E strand of ICAM-4.

7. The epitope according to claim 1, in which the integrins are αᵥ integrins (for example, as found on HT1080 cells), α₄β1 (also known as VLA-4; for example, as found on HEL cells and erythroblasts), or a5P1 (for example, as found on erythroblasts).

8. A footprint domain for binding integrins, comprising a first epitope as defined in claim 1 and a second epitope comprising the C and F strands of domain 1 of ICAM-4 and the CE loop of domain 2 of ICAM-4, in which the C strand (SEQ ID NO: 7) is defined by amino acid residues 47 to 54 of ICAM-4, the F strand (SEQ ID NO: 4) is defined by amino acid residues 77 to 87 of ICAM-4 and the CE loop (SEQ ID NO: 8) is defined by amino acid residues 150 to 158 of ICAM-4, or a functional homologue of the footprint domain.

9. The footprint domain according to claim 8, in which the second epitope is defined by amino acid residues R52 on the C strand of ICAM-4, W77 and L80 on the F strand of ICAM-4, T91, W93 and R97 on the G strand of ICAM-4, and El51 and T154 on the C′-E loop of ICAM-4.

10. The footprint domain according to of claim 8, in which the integrin ligands are ax integrins (for example, as found on HT1080 cells), VLA-4 (for example, as found on HEL cells) and/or the β₂-family of integrins (such as Mac-1, for example, as found on leucocytes and on neutrophils, and/or LFA-1), including αLβ2 (for example, as found on neutrophils).

11. An antagonist of the epitope of claims 1.

12. An antagonist of a ligand for the epitope of claims 1.

13. The antagonist of claim 12, having or consisting essentially of three, four, five, six, seven, eight, nine or more amino acid residues of the A, C, F or G strands or the CE loop of ICAM-4, or a functional homologue thereof.

14. The antagonist of claim 12, in which the antagonist has or consists essentially of the amino acid sequence according to SEQ ID NO: 9, SEQ ID NO: 10 or SEQ ID NO: 11.

15. A method of antagonising the epitope of claims 1, comprising the step of contacting the epitope with an antagonist of the epitope for binding integrins.

16. A method of antagonising a ligand of the epitope of claims 1, comprising the step of contacting the ligand with an antagonist of a ligand of the epitope for binding integrins.

17. A method of treating a disease using the antagonist of claim 11.

18. The method according to claim 17, in which the disease involves ICAM-4.

19. A method of making a medicament for the treatment of a disease comprising the antagonist according to claim 11, wherein the disease involves ICAM-4.

20. The method according to claim 17, in which disease is characterised by increased levels of ICAM-4 binding.

21. The method according to claim 17, in which the disease is characterised by decreased levels of ICAM-4 binding.

22. The method according to claim 17, in which the disease is sickle cell disease, deep vein thrombosis (DVT), malaria, heart disease, vascular complications, diabetes, β-thalassemia, or a thrombotic complication of haematological diseases.

23. An isolated nucleotide encoding the epitope defined in claims 1 or the an antagonist thereof.

24. The isolated nucleotide of claim 23, having a sequence defined within the sequence of SEQ ID NO: 12.
